(19) United States
(12) Patent Application Publication
(10) Pub. No.: US 2022/0193955 A1
(43) Pub. Date: Jun. 23, 2022

(54) PROCESS FOR MAKING COMPOUNDS USING WASTES OF NATURAL ORIGIN AND FIBRES OF PLANT OR ANIMAL ORIGIN

(71) Applicant: MIXCYCLING SRL, Vicenza (IT)

(72) Inventor: Gianni TAGLIAPIETRA, Breganze (IT)

(73) Assignee: MIXCYCLING SRL, Vicenza (IT)

(21) Appl. No.: 17/425,905

(22) PCT Filed: Mar. 6, 2020
(86) PCT No.: PCT/EP2020/056024

§ 371 Date: Jul. 26, 2021

(30) Foreign Application Priority Data
Mar. 12, 2019 (IT) 102019000003565

(51) Int. Cl. B29B 7/92; B29B 7/90; B29C 48/40; B29C 48/345; B29B 9/14

(52) U.S. Cl. CPC B29B 7/92; B29B 7/905; B29B 9/14; B29C 48/345; B29C 48/40

(57) ABSTRACT

The invention concerns a process for making compounds using wastes of natural origin and fibres of plant or animal origin, wherein wastes of natural origin, fibres of plant origin, as well as wastes of animal origin, composing the so-called “charge”, are mixed with agglomerating plastic materials, the so-called “carriers”, and with agglomerating additives in order to form a mixture, the so-called “blend”, which is transformed into a compound, the “compound”, used for making semi-finished products. Said process occurs five sequenced steps that goes from the preparation of the “charge” and the “blend” to the selection of the “carriers” and agglomerating additives till the process of the “blend” to obtain the “compound” and finally to the process of the “compound” to obtain a semi-finished product. According to the invention the initial “charge” is submitted to a sanitization treatment, based on the principle of advanced oxidation, obtained by applying the technological process referred to as “Non-thermal plasma” or

“NTP”, where the so-called “non-thermal discharges with dielectric-barrier method” or “DBDs” are used, in order to strongly reduce bacterial charges, until removal thereof, decompose the volatile organic substances (VOCs) and remove smells.

The present invention relates to a process for making compounds using wastes of natural origin and fibres of plant or animal origin, according to the general part of claim 1.

As is well known in the industrial field, the management of processing wastes of natural origin, in particular those consisting of fibres of animal origin such as, for example, hides and those consisting of fibres of plant origin such as, for example, cork, wood, straw, cereal bran, spent coffee, hemp, cellulose, bamboo fibres, shells of walnuts and hazelnuts, pomace, rice husks for which it is difficult to find a suitable reuse as biomass, so they become waste products, with the consequent economic and ecological difficulties that arise for their disposal.

In the current state of the art, one method for recovering wastes is the one that envisages agglomerating the mass with adhesives, or with plastic materials, to obtain materials and semi-finished products that are used in various branches of industry.

Semi-finished products consisting of slabs obtained with wood-cellulosic residues are mentioned for exemplary purposes which, in the construction sector, are used as thermal insulators, while in the furniture sector they replace the portions normally made of solid wood.

In practice, the products currently on the market, known with the generic term “compound”, obtained by agglomerating the material deriving from wastes or natural fibres with adhesives of various types, show drawbacks that limit their use.

Specifically, a first drawback is constituted by the fact that, in order to avoid the disintegration of the material, it is necessary to use a little “charge”, that is to use a limited quantity of waste product or fibre, which entails the impossibility of making medium- and large-sized products, that is blocks or bales.

A second drawback derives from the fact that the semi-finished products obtained by agglomeration of wastes and natural fibres are considered to be commercially of low value, therefore low-quality adhesives are normally used to contain the cost and are not subjected to optimal sanitization process.

The well-known “pellets” are mentioned, used as domestic fuel for exemplary purposes which, for economic reasons, are made up of various wastes of plant fibres, from wood to cereals, which are agglomerated with poor quality adhesive, so when they burn emit harmful and polluting fumes.

In the documents EP 2565004 A1 and DE 102013101667 A1, which constitute the most relevant documents of the state of the art, processes are described for making compounds using wastes of natural origin and fibres of plant or animal origin, all according to the general part of the claim 1.

The documents US 2006/257299 A1 and JP 2001239239 A1 describe processes that use the technological process called “Non-thermal plasma” or “NTP”, for the treatment of gaseous emissions to eliminate bad odours from industrial contaminants and organic wastes, respectively.

The scientific disclosure of the Bristol Institute of Physics under the title “Plasmas for environmental issues: from hydrogen production to 2D materials assembly” in the of Teterova et al. describes a process that uses “Non-thermal plasma” to remove contaminants from wastewater.

The scientific disclosure having the title “Innovative non-thermal plasma disinfection process inside sealed bags: assessment of bactericidal and sporicidal effectiveness in regard to current sterilization norms” in the name of Zouhaier Ben Belgacem et al. is also mentioned.

The purpose of the present invention is to provide a process for processing wastes of natural origin and fibres of plant or animal origin, of the type which provides for the agglomeration of the components which is free from the drawbacks manifested by similar processes of known type.

In particular, a purpose of the invention is to provide a process for making agglomerates based on natural, animal and plant wastes and fibres, known with the term “compound”, with which it is possible to obtain semi-finished products that have physical characteristics and better mechanical properties than similar products of known type.

This purpose is achieved with the implementation of a process according to the characterizing part of claim 1.

In the dependent claims, particular embodiments of this process are provided.

The whole is better defined by the description of the processing steps of the process according to the invention.

The complete process of the invention substantially comprises the following steps in succession:
step I: preparation of the “charge”;
step II: selection of the carriers and agglomerating additives;
step III: preparation of the blend;
step IV: processing the blend to obtain the compound;
step V: processing the compound to obtain a semi-finished product.

Specifically, each step provides for the following operations:

step I: preparation of the “charge”
1.1 Recovery of the wastes of natural origin and plant or animal fibres which results in the “charge” to be processed;
1.2 Drying of the waste material recovered to give the mass a consistency suitable for the subsequent processing;
1.3 Milling/grinding of the waste material to take the shredded mass to dimensions suitable for the subsequent processing;
1.4 Dimensional sieving to divide the shredded material into various sizes based on the mechanical properties and on the different aesthetic effects that the final compound must have (by way of example, the series of sizes can have dimensions <0.2 mm.; 0.2-0.5 mm.; 0.5-1.0 mm.; 1.0-2.0 mm.; 2.0-4.0 mm);
1.5 Gravimetric sieving to separate, within a same dimensional size, elements with different weight, the lighter ones from the heavier ones, based on the mechanical properties and on the different aesthetical effects that the final compound must have; (by way of example the waste product known as “cork flour” can contain cork particles or bark particles which, although having the same dimensions as the particles of cork flour, have considerably different characteristics);
1.6 Sanitization, a fundamental step due to the fact that a material subject to bacterial contamination is processed, which is based on the principle of advanced oxidation which is carried out through the innovative technology called “Non-thermal plasma” or “NTP”, which uses the so-called “non-thermal discharges with dielectric-barrier method “or” DBD”.

Operationally, the ionization potential and the density of the charge species generated by the plasma with electric barrier charge or “DBD” are greater than those present in the non-thermal plasma generated with other systems, for which a beneficial treatment is achieved which strongly reduces, until elimination, the bacterial charges, the volatile organic substances (VOC) are decomposed and the odours are eliminated, and in addition, in all this, no chemical product is used, thus giving rise to a totally ecological treatment.

Step II: Selection of the Carriers and Agglomerating Additives
2.1 Carriers, plastic materials used to agglomerate natural fibres such as to obtain a well cohesive compound, composed of non-biodegradable plastics, both of fossil origin and bio-based (Polyethylene, Polypropylene, Polystyrene, PMMA, PET, SBS rubbers and SEBS, abs, methacrylate are mentioned for exemplary purposes); or biodegradable plastics, both of fossil origin and bio-based (PLA (polylactic acid) is mentioned for exemplary purposes), plastics derived from corn starch, potatoes, vegetable oils;
2.2 Additives with agglomerating function, composed of modified polymeric adhesives having the function of grafting and considerably increasing the adhesion between the plant or animal material (charge) and polymers (carriers), such as to allow to introduce a higher amount of fibres into the blend and with antibacterial function, based on silver ions.
Operationally, a PLA (polylactic acid) modified with MAH (maleic anhydride) is used with biodegradable materials, while with non-biodegradable materials, polymers modified based on PP (polypropylene), SEBS (styrene-ethylene-butylene-styrene), PE (polyethylene in all forms thereof), all grafted with MAH (maleic anhydride) are used.

### Step III: Preparation of the Blend

mixture consisting of the charge, carriers and agglomerating additives in which the percentages of the three components vary based on the type of grafting wanted and the mechanical/physical characteristics that the compound or the semi-finished final product must have; operationally, the blend is a mixture that can comprise: charges from 10% to 80%—carriers from 20% to 90%—additives from 1% to 5%, in the various combinations.
   step IV: Processing the Blend to Obtain the Compound
4.1 Preparation of the raw materials (charges, carriers, agglomerating additives) by means of molecular-sieve dehumidifiers, able to ensure a correct humidity down into the inside of the particles (granules or fibres), in order to increase adhesion between the components and prevent the creation of condensation during the compound processing steps;
4.2 Mixing: the various components which are “dry blend” mixed with specific mixers or, in alternative, using volumetric or gravimetric dosing devices which mix the various components directly inside the machine.
   step V: Processing the Compound to Obtain a Semi-Finished Product
5.1 Extrusion: a specific twin-screw extruder which mixes homogeneously the components, without stressing or burning fibres, and, by means of a drawing head, makes filaments/strands with a diameter comprised between 3 mm and 5 mm which are, through a cutter, shredded into granules (pellets) and, thereafter, are sieved to be divided based on the weight or on predefined sizes;
5.2 Sanitization of the granules: it is carried out through the innovative technology called

“Non-thermal Plasma” or “NTP”, which uses the so-called “non-thermal discharges with dielectric-barrier method” or “DBD”, equal to the one used and described for sanitizing the “charge”;
5.3 Packaging: pellets are packaged in protection bags under “NTP”-modified atmosphere (Non Thermal-Plasma), to make the product more salubrious and increase its “shelf life”.

1-7. (canceled)

8. Process for making compounds using wastes of natural origin and fibres of plant or animal origin, of the type wherein wastes of natural origin, fibres of plant origin, as well as wastes of animal origin, composing the so-called “charge”, are mixed with agglomerating plastic materials, the so-called “carriers”, and with agglomerating additives in order to form a mixture, the so-called “blend”, which is transformed into a compound, the “compound”, used for making semi-finished products, said process providing the following sequenced steps:
step I: preparation of the “charge”;
step II: selection of the “carriers” and agglomerating additives;
step III: preparation of the “blend”;
step IV: processing the “blend” to obtain the “compound”;
step V: processing the “compound” to obtain a semi-finished product,
wherein
the initial “charge” is submitted to a sanitization treatment, based on the principle of advanced oxidation, obtained by applying the technological process referred to as “Non-thermal plasma” or “NTP”, where the so-called “non-thermal discharges with dielectric-barrier method” or “DBDs” are used, in order to strongly reduce bacterial charges, until removal thereof, decompose the volatile organic substances (VOCs) and remove smells.

9. PROCESS, according to claim 8, wherein the final semi-finished product obtained with the “compound” is submitted to a sanitization treatment, based on the principle of advanced oxidation, obtained by applying the technological process referred to as “NTP”, where the so-called “DBDs” are used.

10. PROCESS, according to claim 8, wherein the step I comprises the following processing:
recovery of the wastes of natural origin and plant or animal fibres, from the factories producing wastes and which results in the “charge”;
drying of the waste material recovered to give the mass a consistency suitable for the subsequent processing;
milling/grinding of the waste material to take the shredded mass to dimensions suitable for the subsequent processing.
dimensional sieving to divide the shredded material into various sizes based on the mechanical properties and on the different aesthetic effects that the final “compound” must have;
gravimetric sieving to separate, within a same dimensional size, elements with different weight, the lighter ones from the heavier ones, based on the mechanical properties and on the different aesthetical effects that the final “compound” must have;
sanitization, to strongly reduce the bacterial charges, until removal thereof, decompose the volatile organic substances (VOCs) and remove smells.

11. PROCESS, according to claim 8, wherein the step II comprises the following processing:
selection of the “carriers”, plastic materials used to agglomerate natural fibres such as to obtain a well cohesive “compound”, composed of non-biodegradable plastics, both of fossil origin and bio-based (Polyethylene, Polypropylene, Polystyrene, PMMA, PET, SBS rubbers and SEBS, abs, methacrylate are mentioned for exemplary purposes), or biodegradable plastics, both of fossil origin and bio-based (PLA (polylactic acid) is mentioned for exemplary purposes), plastics derived from corn starch, potatoes, vegetable oils;
selection of the additives: those with agglomerating function, composed of modified polymeric adhesives having the function of grafting and considerably increasing the adhesion between the plant or animal material (charge) and polymers (carriers), such as to allow to introduce a higher amount of fibres into the “blend” and where a PLA (polylactic acid) modified with MAH (maleic anhydride) is used with biodegradable materials, while with non-biodegradable materials, polymers modified based on PP (polypropylene), SEBS (styrene-ethylene-butylene-styrene), PE (polyethylene in all forms thereof), all grafted with MAH (maleic anhydride) and those with antibacterial function, based on silver ions, are used.

12. PROCESS, according to claim 8, wherein the step III consists in preparing the “blend”, a mixture composed of the “charge”, “carriers” and agglomerating additives, where the percentages of the three components vary based on the intended type of graft and on the mechanical/physical features that the “compound” or the final semi-finished product must have;

13. PROCESS, according to claim 8, wherein the step IV, consisting in processing the “blend” to obtain the “compound”, includes the following steps:
preparation of the raw materials (charges, carriers, agglomerating additives) by means of molecular-sieve dehumidifiers, able to ensure a correct humidity down into the inside of the particles (granules or fibres), in order to increase adhesion between the components and prevent the creation of condensation during the “compound” processing steps;
mixing of the various components which are “dry blend” mixed with specific mixers or, in alternative, using volumetric or gravimetric dosing devices which mix the various components directly inside the machine.

14. PROCESS, according to claim 8, wherein the step V, which consists in processing the “compound” to obtain a semi-finished product, includes:
extrusion by means of a twin-screw extruder which mixes homogeneously the components, without stressing or burning fibres, and, by means of a drawing head, makes filaments/strands which are shredded into granules (pellets) and, thereafter, are sieved to be divided based on the weight or on predefined sizes;
sanitization of the granules which is based on the principle of advanced oxidation, obtained by applying the technological process referred to as “NTP”, where the so-called “DBDs” are used.
packaging of pellets in protection bags under “NTP”-modified atmosphere, to make the product more salubrious and increase its “shelf life”.
